(19) United States
(12) Patent Application Publication
(10) Pub. No.: US 2013/0184751 A1
(43) Pub. Date: Jul. 18, 2013

(54) SPINOUS PROCESS IMPLANT WITH PLATE MOVEABLE BY GEAR NUT

(71) Applicant: Daniel E. Siegfried, Memphis, TN (US)

(72) Inventor: Daniel E. Siegfried, Memphis, TN (US)

(73) Assignee: Warsaw Orthopedic, Inc., Warsaw, IN (US)

(21) Appl. No.: 13/351,406

(22) Filed: Jan. 17, 2012

(51) Int. Cl. A61B 17/70; A61B 17/88

(52) U.S. Cl. 606/248; 606/279

(57) ABSTRACT

A spinal implant with two plates that are connected together by a post, with the plates configured to be positioned on outer lateral sides of spinous processes with the post extending through the interspinous space. The second of the two plates includes a bore that receives the post, and that plate is movable along the length of the post, and selectively lockable in position. A gear nut is threadable on a distal section of the post and includes external gear teeth that are used to drive the gear nut so as to move the second plate toward the first plate. When the plates are in their desired locations, the second plate is locked in position. The gear nut may or may not then be removed. The use of the gear nut facilitates one or more of assembly, insertion, and affixation of the spinal implant. Related methods are also disclosed.

### BACKGROUND

The present invention generally relates to devices and methods for stabilizing vertebral members, and more particularly, to spinal implants that mount onto the spinous processes.

Vertebral members typically comprise a vertebral body, pedicles, laminae, and processes. The processes are projections that serve as connection points for the ligaments and tendons, and typically include the articular processes, transverse processes, and the spinous process. Intervertebral discs are located between adjacent vertebral bodies to permit flexion, extension, lateral bending, and rotation.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion. One manner of correcting the damage involves mounting of a spinal implant onto the spinous processes, typically in association with a fixation process such as anterior lumbar interbody fusion (ALIF), posterior lumbar interbody fusion (PLIF), intertransverse lumbar interbody fusion (ILIF), and the like. See, for example, the spinal implant sold under the trade name CD HORIZON SPIRE™ by Medtronic Spinal and Biologics of Memphis, Tenn., and the devices described in U.S. Pat. Nos. 7,048,736 and 7,727,233. While these devices provide some solutions, they may not be ideal for some situations. As such, there remains a need for alternative spinal implants and related methods.

### SUMMARY

The present application is directed to implants for attaching to spinous processes. In one or more embodiments, the implant includes a first plate sized to extend along a first lateral side of the spinous processes and a second plate sized to extend along a second lateral side of the spinous processes. The second plate includes a first bore that extends through the second plate between a medial surface that faces towards the first plate and an opposing outer surface. An elongated post extends along a post longitudinal axis from a proximal section attached to the first plate to a distal section positioned distally from the first plate. The post distal section extends through the first bore and has an external thread thereon. The first bore is sized relative to the post for the second plate to be infinitely movable along the post and selectively lockable in position therealong. A gear nut is mountable on the post. The gear nut has an internal thread for threadably engaging with the external thread of the post. The gear nut has external gear teeth on a periphery thereof. The gear teeth may have a generally triangular profile, or other suitable gear profile. The gear nut and the post are configured such that, with the gear nut mounted on the post, rotation of the gear nut about the post axis in a first direction narrows a maximum spacing between the first and second plates. The second plate may include a fastener bore intersecting the first bore, and the implant may further include a fastener disposable in the fastener bore and configured to be movable relative to the second plate to selectively lock the second plate relative to the post. The fastener may be a setscrew. The gear nut may include a proximal flange that extends farther out away from the post axis than the gear teeth and bears against the outer surface of the second plate. The gear nut may include a collar section disposed proximally relative to the gear teeth, with the collar section disposed between the post and the second plate. The collar section may be clampingly engaged by a locking fastener to selectively lock the second plate relative to the post. The post and gear nut may be formed of the same material. The outer surface of the second plate may have a flat recessed surface disposed normal to the post axis, with the gear nut bearing against the flat recessed surface. The first and second plates may each comprise a plurality of protrusions extending toward each other, with the protrusions configured to bite into the spinous processes.

The present application is also directed to methods of attaching an implant to spinous processes. In one or more embodiments, the methods may include positioning a first plate on a first lateral side of the spinous processes and positioning a second plate on a second lateral side of the spinous processes in spaced relation to the first plate. A post that extends outward from the first plate is positioned through an interspinous space formed between the spinous processes and through a bore in the second plate. The post has a post longitudinal axis and an external thread on a distal section thereof. A gear nut is threadably engaged on the post distal section with the second plate disposed between the gear nut and the first plate. The gear nut has an internal thread and external gear teeth. The method includes displacing the second plate along the post axis toward the first plate by driving the external gear teeth of the gear nut so as to rotate the gear nut in a first direction about the post axis. Thereafter, the second plate is locked relative to the post. The gear nut may include a collar section disposed proximally relative to the gear teeth and in the bore, and the locking the second plate relative to the post may include tightening a fastener against the collar section. The method may include threading the gear nut onto the post distal section, with the second plate disposed between the gear nut and the first plate, prior to positioning the first plate, positioning the second plate, and positioning the post. Driving the external gear teeth of the gear nut may include removably coupling an instrument to the gear nut and driving the external gear teeth via the instrument to rotate the gear nut. The removably coupling the instrument to the gear nut may include moving the instrument toward the gear nut normal the post axis. The gear nut may include a proximal flange that extends farther out away from the post axis than the gear teeth, and the proximal flange may press the second plate toward the first plate during the driving of the external gear teeth. The method may include engaging a fastener with the second plate after the rotating the gear nut and prior to the locking, and the locking the second plate may include advancing the fastener toward the post axis. The method may include pivoting the post relative to the first plate prior to locking the second plate relative to the post. The method may include thereafter removing the gear nut from the post by rotating the gear nut in a second direction opposite the first direction. The method may be such that displacing the second plate toward the first plate includes causing protrusions on the first and second plates to bite into opposing lateral sides of adjacent spinous processes.

The various aspects of the various embodiments may be used alone or in any combination, as is desired.

### BRIEF DESCRIPTION OF THE DRAWINGS

FIG. 1 shows a spinal implant according to one embodiment mounted to a spinal column.

FIG. 2 shows a perspective view of the spinal implant of FIG. 1 with the gear nut attached.

FIG. 3 shows a partially exploded perspective view of the spinal implant of FIG. 1 with the gear nut detached, and a surgical instrument.

FIG. 4 shows a top (posterior to anterior) view of the implant of FIG. 2, prior to tightening of the gear nut.

FIG. 5 shows an end view of the implant of FIG. 4 after the gear nut is advanced to narrow the distance between the plates, and with a surgical tool engaged with the gear nut.

FIG. 6 shows an end view of the implant of FIG. 5 after tightening of the locking fastener to lock the second plate relative to the post, and the surgical tool removed.

### DETAILED DESCRIPTION

In one embodiment, the present application is directed to a spinal implant with two plates that are connected together by a post. The implant is configured for each plate to be positioned on outer lateral sides of spinous processes with the post extending through the interspinous space. The second of the two plates includes a bore that receives the post, and that plate is movable along the length of the post to accommodate different anatomies such as for relatively wide or thin spinous processes, and selectively lockable in position. A gear nut is threadable on a distal section of the post. The gear nut includes external gear teeth that are used to drive the gear nut so as to move the second plate toward the first plate. When the plates are in their desired locations, the second plate is locked in position. The gear nut may then be removed, or may be left in place. The use of the gear nut facilitates one or more of assembly, insertion, and affixation of the spinal implant.

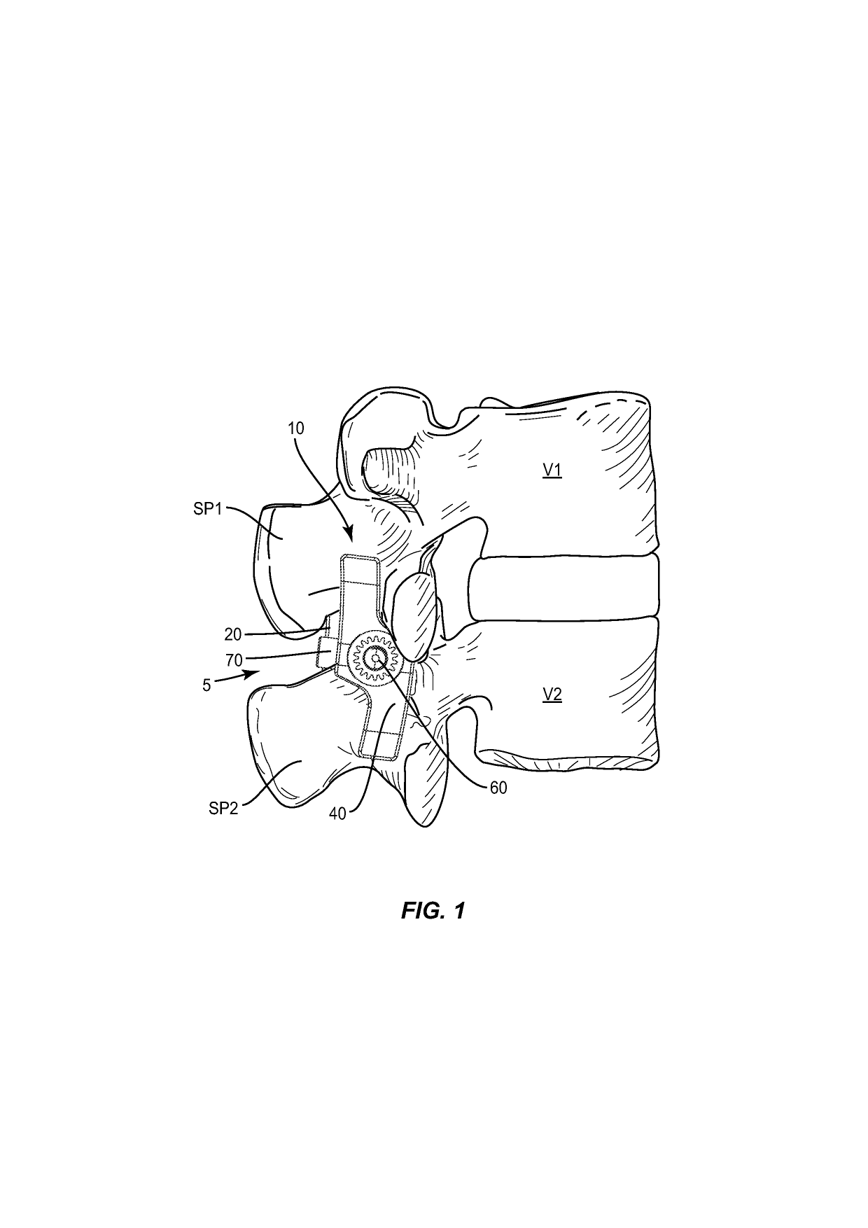

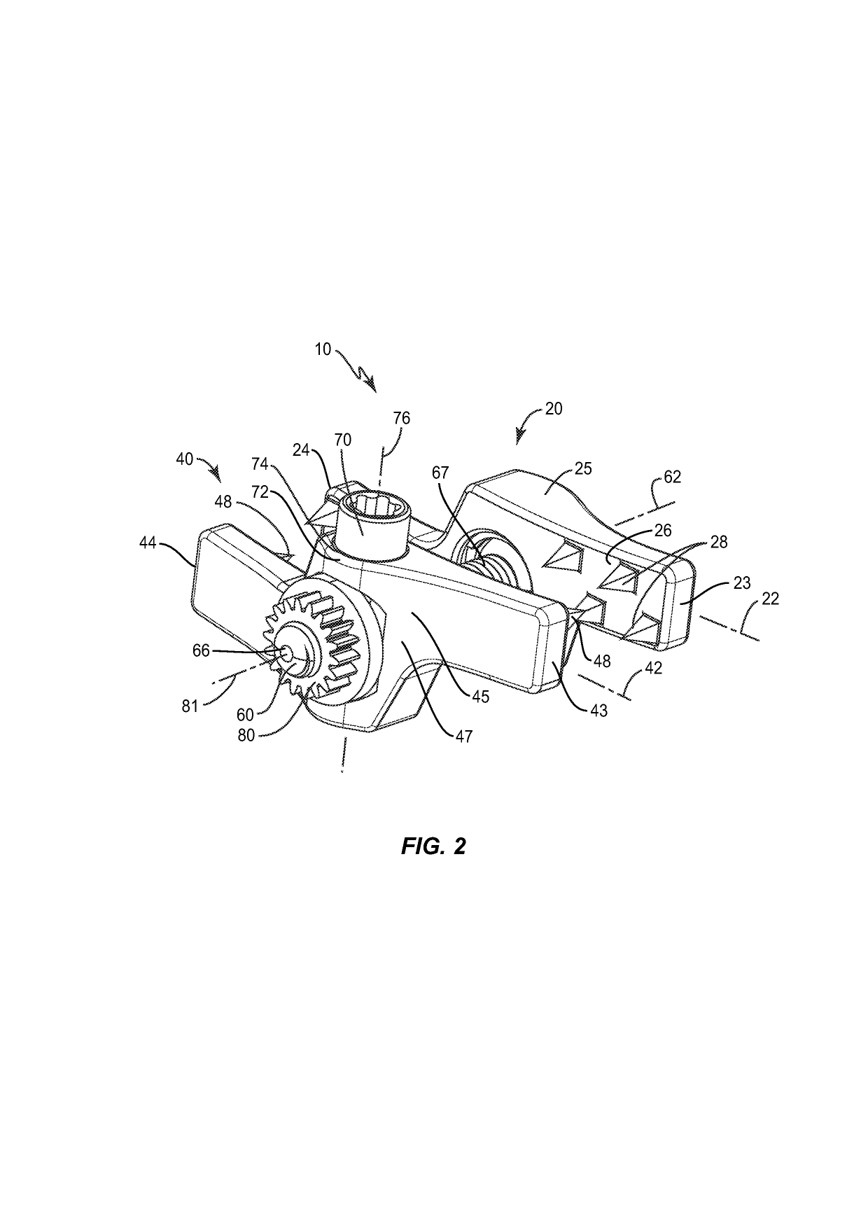

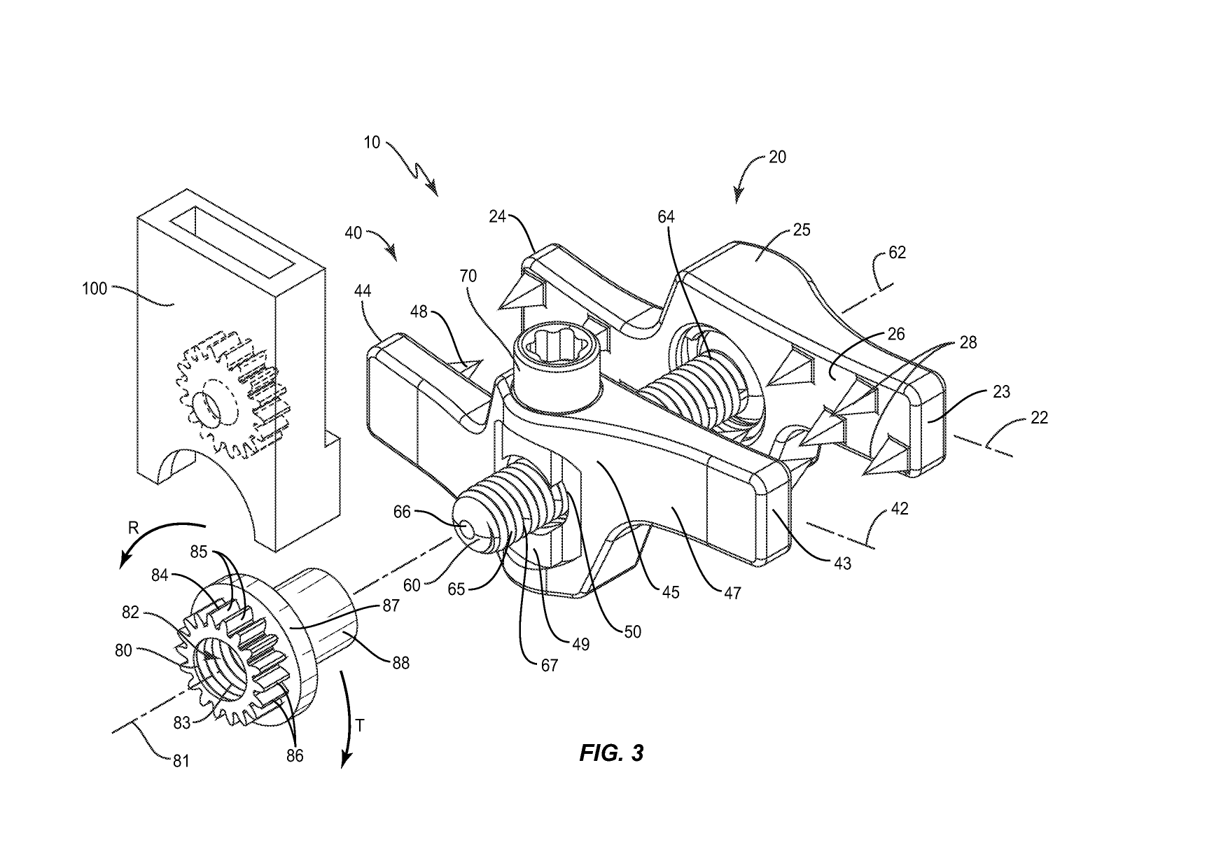

Referring to FIG. 1, a spinal implant according to one embodiment and generally designated 10 is shown clampingly mounted to the spinous process SP1 of a superior vertebra V1 and a spinous process SP2 of an inferior vertebra V2. A portion of the implant 10, in particular post 60, extends transversely through the interspinous space 5 between the two spinous processes SP1,SP2. The implant 10, shown more clearly in FIGS. 2-5, includes a first plate 20, a second plate 40, an interconnecting post 60, a fastener 70, and a gear nut 80.

Referring to FIGS. 2-4, the first plate 20 may be elongate along an associated longitudinal axis 22, with a superior end section 23, an inferior end section 24, and an intermediate section 25. If desired, the superior end section 23 and inferior end section 24 may be shifted in an anterior direction or a posterior direction so that the first plate has a somewhat Z-shape as shown, although this is optional and the first plate 20 may be generally rectilinear or any other suitable shape as is desired. The first plate 20 has a length sufficient to vertically span the interspinous gap 5 (interspinous space) between adjacent spinous processes while substantially overlapping the spinous processes SP1,SP2. The first plate 20 has a medial face 26 and an opposite lateral face 27. The medial face 26 includes a plurality of protrusions 28 that extend medially for biting into the corresponding spinous process SP1,SP2. Advantageously, the protrusions take the form of a plurality of sharp teeth. The teeth 28 may advantageously be disposed in two groups, one on the superior end section 23 and one on the inferior end section 24, with the intermediate section 25 being free of such teeth 28. The lateral face 27 may have suitable features, such as recesses or the like, for cooperating with installation and manipulation instrumentation. The tips of superior end 23 and inferior end 24 are advantageously generally rounded so as to minimize damage to surrounding tissue and for ease of installation.

The second plate 40 may be substantially similar to the first plate 20. For example, the second plate 40 may be elongate along an associated longitudinal axis 42, with a superior end section 43, an inferior end section 44, and an intermediate section 45. If desired, the second plate 40 may have a somewhat Z-shape similar to the first plate 20, or may be any other suitable shape as is desired. The second plate 40 advantageously has a length sufficient to vertically span the interspinous gap 5 while substantially overlapping the spinous processes SP1,SP2. The second plate 40 has a medial face 46 and an opposite lateral face 47, with the medial face 46 facing the medial face 26 of the first plate 20. The medial face 46 includes a plurality of protrusions 48 similar to teeth 28 for biting into the spinous processes SP1,SP2. The lateral face 47 may have suitable features, such as recesses or the like, for cooperating with installation and manipulation instrumentation. The tips of superior end section 43 and inferior end section 44 are advantageously generally rounded so as to minimize damage to surrounding tissue and for ease of installation. The intermediate section 45 may have a suitable boss 72 thereon, with a hole 74 having centerline 76 for receiving the fastener 70, as discussed below. The intermediate section 45 of the second plate 40 includes a bore 50 that extends from medial face 46 to lateral face 47, through the intermediate section 45. The bore 50 is sized to receive post 60 and a surrounding portion of gear nut 80, and therefore has a cross-section at least as large, and advantageously larger than the corresponding portion of gear nut 80, as described further below.

The post 60 may take the form of a round shaft that extends along a post longitudinal axis 62 from a post proximal section 64 proximate the first plate 20 to a post distal section 65 proximate the second plate 40. The post 60 has a length sufficient to extend laterally across the interspinous gap 5 and through the bore 50 of second plate 40. In some embodiments, the post proximal section 64 is mounted to the first plate 20 by any suitable means, such as welding or the like. Alternatively, the post 60 may be integrally formed with the first plate 20. Still further, the post 60 may be pivotally attached to the first plate 20. For example, the post 60 may be pivotally attached either for monoaxial or polyaxial movement relative to the first plate 20 about one or more pivot axes, such as about a pivot axis perpendicular to the post axis 62 and parallel to medial face 26. Examples of suitable pivoting structures are shown in U.S. Pat. Nos. 7,048,736 and/or 7,727,233. The post 60 may include flats or other features (not shown) for engaging with the fastener 70. The post 60 may advantageously be rigid and generally solid. The distal section 65 terminates at a distal tip 66. The post distal section 65 advantageously has a cylindrical cross-sectional shape and includes external thread 67. Thread 67 extends proximally a significant distance, and may extend all the way to or into the proximal section 64.

The fastener or locking member 70 may take the form of a simple setscrew, optionally with tapered tip, that is sized to threadably engage hole 74 in second plate 40. When tightened, the locking member 70 presses against a portion of gear nut 80 to lock the relative distance between the plates 20,40. Of course, other forms of fasteners, such as concentrically barbed posts, quarter-turn fasteners, and the like, may alternatively be used.

The gear nut 80 is threadably mounted on post distal section 65. The gear nut 80 includes a central bore 82 that includes internal thread 83 sized and configured to inter-engage with post thread 67. The gear nut 80 also includes external gear teeth 84 disposed around a periphery thereof. The tips 86 of the gear teeth 84 are spaced from each other, with flanks 85 that are advantageously likewise spaced from each other. Any suitable profile of gear teeth 84 may be used, such as triangular, involute, etc. The gear nut 80 may optionally include a flange 87 that is advantageously generally disc-shaped. The flange 87 advantageously extends outward at least as far as the tips 86 of the gear teeth 84. The proximal face of the flange 87 is advantageously smooth. In addition, the gear nut 80 may optionally include a collar section 88 extending proximally from flange 87. The collar 88 is sized and configured to receive the post distal section 65 and be received in bore 50 of second plate 40 and may advantageously be generally cylindrical.

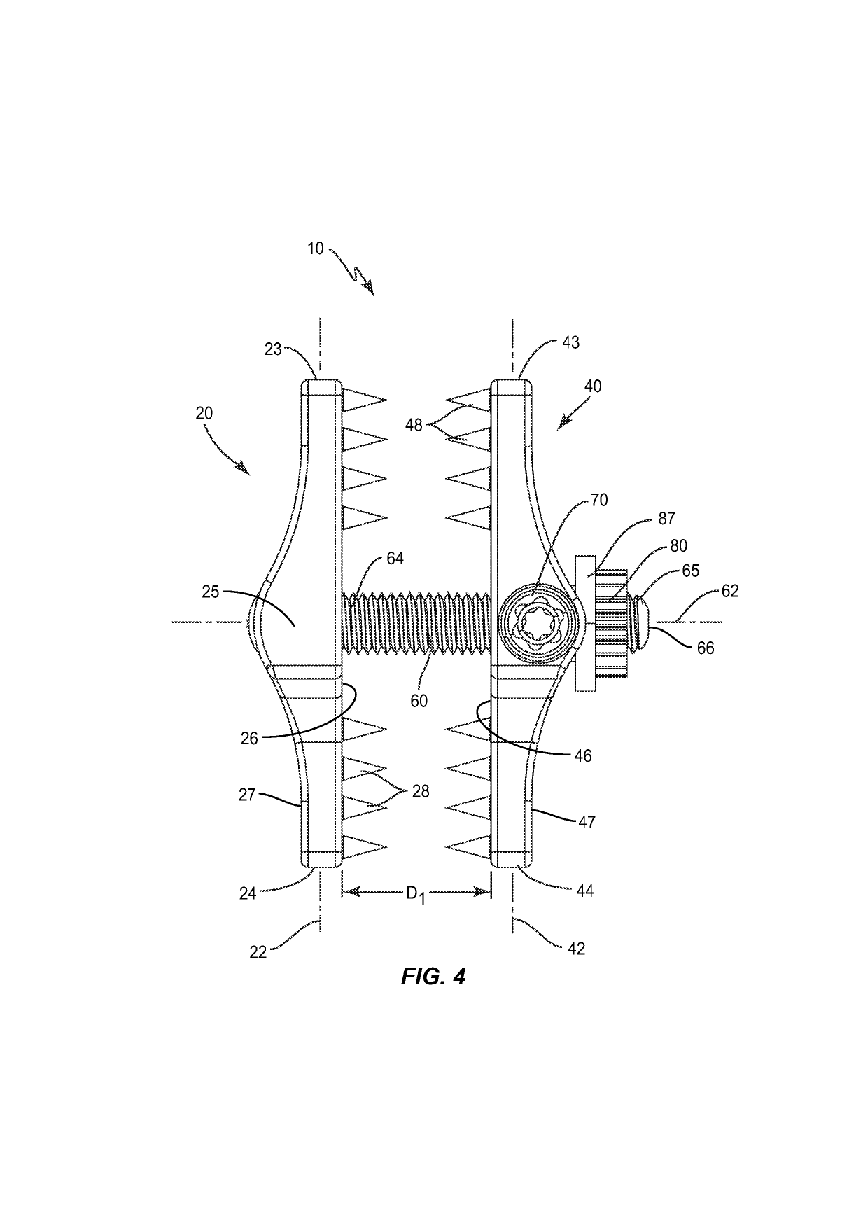

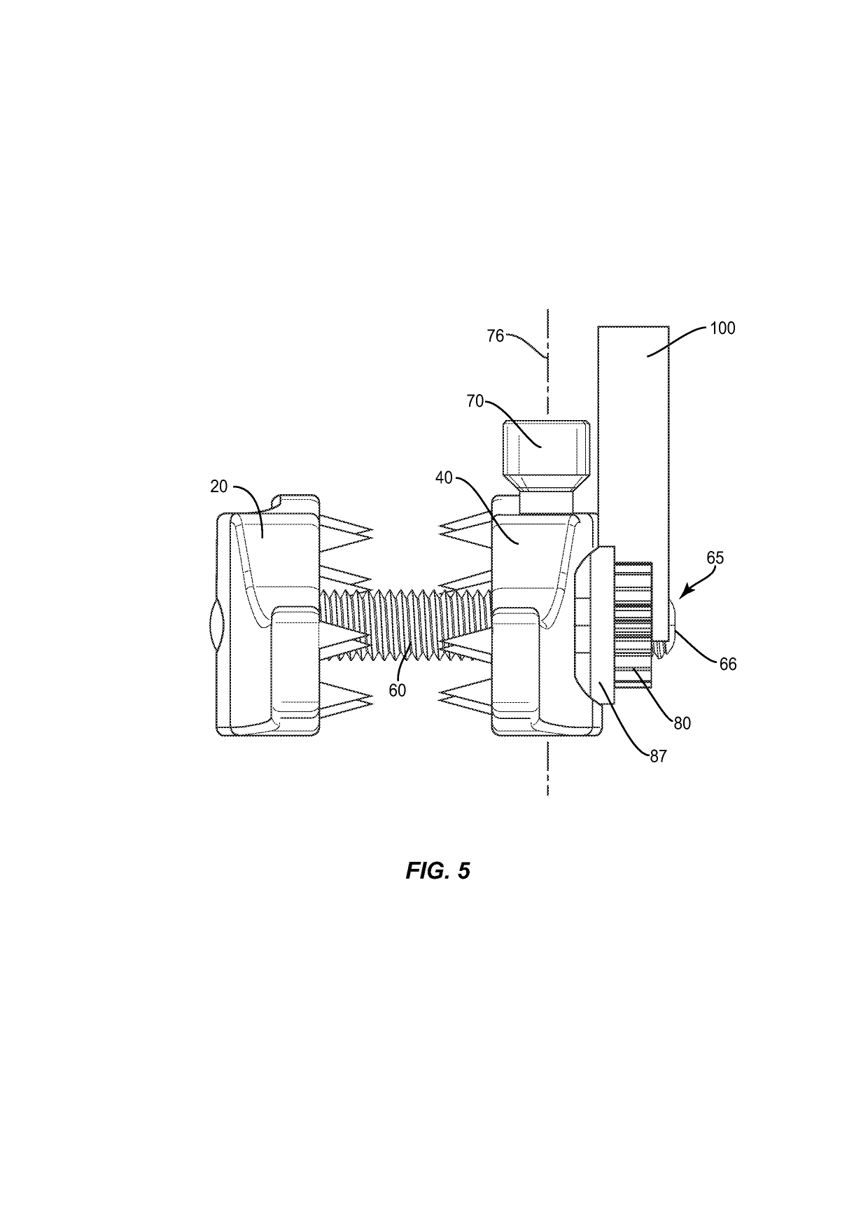

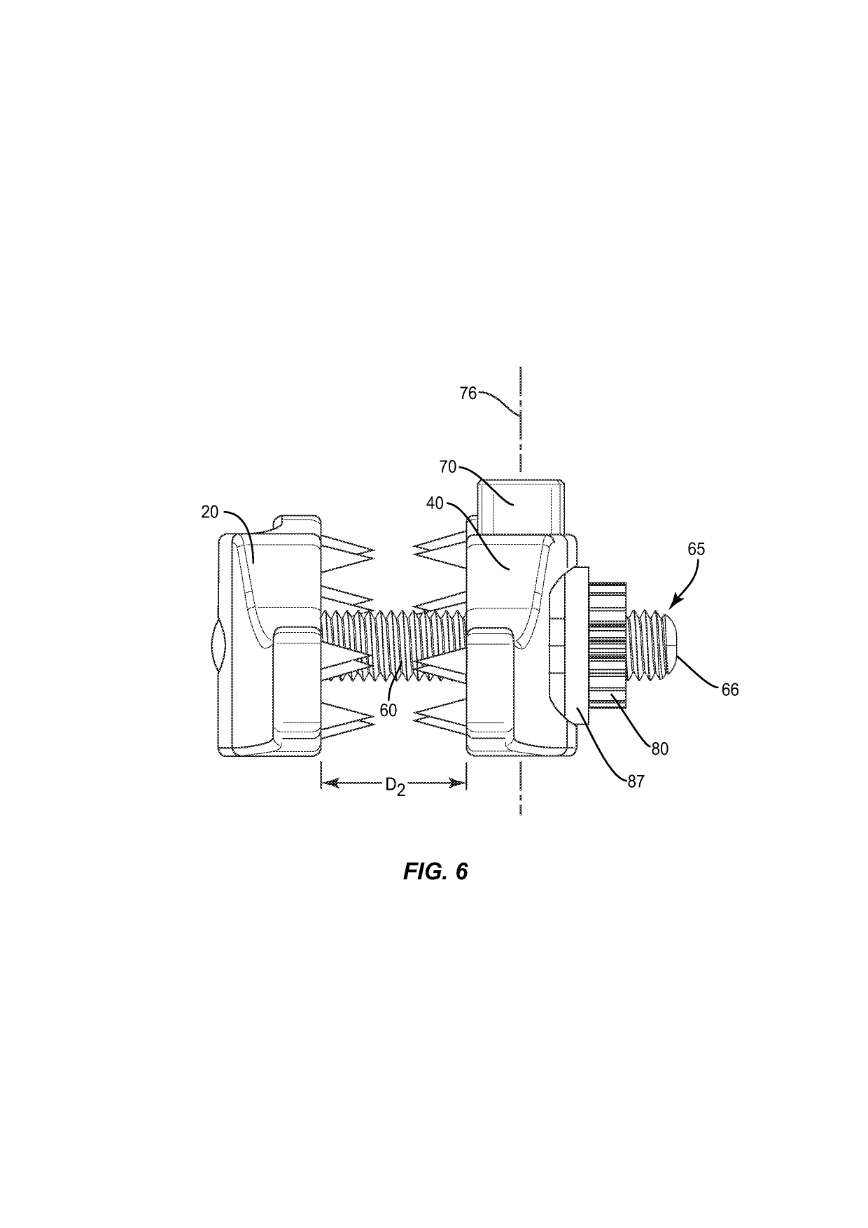

In use, the device 10 can be implanted for posterior spinal stabilization as a stand-alone procedure or in conjunction with other procedures. The device 10 can be positioned through a small posterior incision in the patient of sufficient size to admit the device and instrumentation. Following the incision, muscle is moved aside if and as needed for placement of the device 10. The spinous processes SP1,SP2 are optionally distracted using suitable instrumentation known in the art, and the first plate 20 is implanted such that the superior end section 23 extends on a first lateral side of spinous process SP1, inferior end section 24 extends on the first side of spinous process SP2, and post 60 extends through the interspinous space 5 generally normal to the sagittal plane defined by the spinous processes SP1,SP2. This implantation may involve pivoting of the post 60 in some embodiments. With the first plate 20 in position, the distal tip 66 of post 60 extends laterally beyond the spinous processes SP1,SP2 on the lateral side opposite first plate 20. The second plate 40 may then be added by inserting post 60 into bore 50, and sliding the second plate 40 on post 60 slightly toward the first plate 20. As the second plate 40 is slid toward the first plate 20, the post tip 66 becomes positioned distally beyond the lateral face 47 of the second plate. In other words, the tip 66 of post protrudes out bore 50. Note that the fastener 70 may be threaded into hole 74 at this point in the procedure, but should not be fully tightened so as to allow for movement of the second plate 40 along post (at least parallel to post axis 62, and possibly with additional degrees of freedom). The gear nut 80 is threaded onto the post thread 67, advantageously so that flange 87 bears against lateral face 47 near bore 50 and collar 80 extends into bore 50 around post distal section 65. See FIG. 4. A surgical tool 100 is then used to tighten gear nut 80 and force second plate 40 toward first plate 20. See FIG. 5. The tool 100 includes a drive gear (not shown) that engages the gear teeth 84 and applies a rotational force to the gear nut 80 so as to rotate the gear nut 80 about rotational axis 81 (which is coincident with post axis 62) in a tightening direction T (e.g., clockwise). Due to the inter-engagement of thread 67 with thread 83, rotation of gear nut 80 about post axis 62 in direction T causes the gear nut 80 to advance (move proximally) along post 60 toward the first plate 20, pushing the second plate 40 toward the first plate 20. As can be appreciated, the position of the gear nut 80 along post 60 determines the maximum spacing between the plates 20,40 in that the second plate 40 may, in some embodiments, be closer to the first plate 20 than the limit set by the position of the gear nut 80. Thus, during the tightening of the gear nut, the spacing between the medial faces 26,46 of plates 20,40 changes from, for example, a distance of D₁ (FIG. 4) to a smaller distance D₂ (FIG. 6). Note also, that the second plate 40 is advantageously positionable along post 60 at an infinite number of positions, as the second plate 40 can conceptually slide to any number of positions along post 60 and be locked in the selected position by fastener 70. Thus, the implant 10 is able to accommodate a wide variety of patient morphologies. When the second plate 40 is moved sufficiently close to the first plate 20, such as when the plates 20,40 are clamped in the desired location on the spinous processes SP1,SP2, with teeth 28,48 biting into the opposing lateral sides of spinous processes SP1,SP2, the tool 100 may be disengaged from the gear nut 80 and removed. The fastener 70 is then tightened by advancing fastener 70 to clamp against the collar 88 of gear nut 80 to lock the second plate 40 in position along post 60. See FIG. 6.

Clamping plates 20,40 to the spinous processes SP1,SP2 helps maintain the alignment and spacing of the spinous processes SP1,SP2 while also providing resistance to spinal extension and flexion. Thus, engagement of plates 20,40 to the spinous processes SP1,SP2 resists movement of the spinous processes SP1,SP2 toward and away from one another as a result of spinal extension and flexion, respectively, or as a result of any other movement or condition.

While the above description has been in the context of an in-situ assembly of the first and second plates 20,40, in some embodiments the device 10 may be inserted in an already-assembled condition, with the second plate 40 and gear nut 80 already disposed on the post 60. Thus, the implant 10 may, in some embodiments, be assembled by the manufacturer or by medical personnel, prior to insertion of the implant 10 into the patient.

In some embodiments, the second plate 40 may advantageously include a flat recessed surface 49 that receives the gear nut 80. The surface 49 is advantageously disposed normal to post axis 62 so that second plate 40 is pressed evenly toward first plate 20 when gear nut 80 is tightened. The proximal face of proximal flange 84 advantageously bears against surface 49.

It should be noted that internal threading 83 and external threading 67 is typically a single start threading, but may alternatively be a multiple start threading, such as a dual-start, etc. The term “thread” is intended to cover all such threadings unless modified by a term such as “single” or the like.

The above description has generally been in the context of having the fastener 70 clamping against collar 88 of gear nut 80 to lock the second plate 40 in position. However, in some embodiments, the fastener 70 clamps against post 60 rather than gear nut 80. For such embodiments, the collar 88 may be omitted, and bore 50 down-sized accordingly to better fit post 60. Also, for such embodiments, the gear nut 80 may be removed if desired (and at any point after the plates 20,40 are locked in position and prior to closing the surgical site) by rotating the gear nut 80 in a loosening direction R (e.g., counter-clockwise) until the gear nut 80 is disengaged from the post 60.

As will be appreciated, the first plate 20, second plate 40, post 60, fastener 70, and gear nut 80 may each be made from any suitable biocompatible rigid materials such as titanium and its alloys, stainless steel, cobalt chrome, ceramics, relatively rigid polymers like carbon reinforced polyetheretherketone (PEEK), or the like, known in the art. As can be appreciated, the first plate 20, second plate 40, fastener 70, and gear nut 80 are advantageously distinct (i.e., separate) pieces from each other that are joined together during assembly.

In some embodiments, a sleeve (not shown) may be disposed on post 60 to provide additional support of the vertebrae to maintain or provide post-operative distraction between the spinous processes SP1,SP2. The sleeve may be osteoconductive if desired. For more information on sleeves, see U.S. Pat. No. 7,727,233.

While FIG. 1 shows an implant applied to vertebra L-4 and L-5, the implant device can be implanted on spinous processes at other levels. For example, levels up to T-3 may be appropriate sites. Also, plates 20,40 bridging more than one level may also be considered, optionally with multiple posts 60 and gear nuts 80 disposed at suitable intervals.

As discussed above, the gear nut 80 has external gear teeth 84 thereon. A surface having gear teeth has an intervening point (e.g., a point at the minor diameter) between adjacent teeth that lies closer to the axis of rotation than a line connecting the tips of those two teeth. In the present case, the gear teeth 84 of gear nut 80 have a “valley” formed by the flanks 85 of adjacent teeth that have a multitude of points disposed closer to the axis of rotation (post axis 62) than a theoretical line connecting the adjacent tips 86. In contrast, a conventional hexagonal nut does not have gear teeth as that term is used herein because each of the facets interconnecting the apexes of the hexagon are flat surfaces lying along the line that connects the adjacent apexes.

The implant 10 may be used during surgical procedures on living patients. The implant may also be used in a non-living situation, such as within a cadaver, model, and the like. The non-living situation may be for one or more of testing, training, and demonstration purposes.

All U.S. patents, patent application publications, and applications mentioned above are hereby incorporated herein by reference in their entirety.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

What is claimed is:

1. A spinal implant for attaching to adjacent spinous processes comprising:
a first plate sized to extend along a first lateral side of the spinous processes;
a second plate sized to extend along a second lateral side of the spinous processes, the second plate including a first bore that extends through the second plate between a medial surface that faces towards the first plate and an opposing outer surface;
an elongated post that extends along a post longitudinal axis from a proximal section attached to the first plate to a distal section positioned distally from the first plate; the post distal section extending through the first bore and having an external thread thereon;
the first bore sized relative to the post for the second plate to be infinitely movable along the post and selectively lockable in position therealong;
a gear nut mountable on the post; the gear nut having an internal thread for threadably engaging with the external thread of the post; the gear nut having external gear teeth on a periphery thereof;
wherein the gear nut and the post are configured such that, with the gear nut mounted on the post, rotation of the gear nut about the post axis in a first direction narrows a maximum spacing between the first and second plates.

2. The spinal implant of claim 1:
wherein the second plate further comprises a fastener bore intersecting the first bore;
further comprising a fastener disposable in the fastener bore and configured to be movable relative to the second plate to selectively lock the second plate relative to the post.

3. The spinal implant of claim 2 wherein the fastener is a setscrew.

4. The spinal implant of claim 1 wherein the gear nut includes a proximal flange that extends farther out away from the post axis than the gear teeth and bears against the outer surface of the second plate.

5. The spinal implant of claim 1 wherein the outer surface of the second plate comprises a flat recessed surface disposed normal to the post axis; the gear nut bearing against the flat recessed surface.

6. The spinal implant of claim 1 wherein the first and second plates each comprise a plurality of protrusions extending toward each other; wherein the protrusions are configured to bite into the spinous processes.

7. The spinal implant of claim 1 wherein the gear nut further comprises a collar section disposed proximally relative to the gear teeth; wherein the collar section is disposed between the post and the second plate.

8. The spinal implant of claim 7 wherein, when the second plate is selectively locked relative to the post, a locking fastener clampingly engages the collar section.

9. The spinal implant of claim 1 wherein the gear teeth have a generally triangular profile.

10. The spinal implant of claim 1 wherein the post and the gear nut are formed of the same material.

11. A method of attaching an implant to spinous processes comprising:
positioning a first plate on a first lateral side of the spinous processes;
positioning a second plate on a second lateral side of the spinous processes in spaced relation to the first plate;
positioning a post that extends outward from the first plate through an interspinous space formed between the spinous processes and through a bore in the second plate; the post having a post longitudinal axis and an external thread on a distal section thereof;
wherein a gear nut is threadably engaged on the post distal section with the second plate disposed between the gear nut and the first plate; the gear nut having an internal thread and external gear teeth;
displacing the second plate along the post axis toward the first plate by driving the external gear teeth of the gear nut so as to rotate the gear nut in a first direction about the post axis;
thereafter, locking the second plate relative to the post.

12. The method of claim 11 wherein the gear nut comprises a collar section disposed proximally relative to the gear teeth and in the bore; wherein locking the second plate relative to the post comprises tightening a fastener against the collar section.

13. The method of claim 11 further comprising threading the gear nut onto the post distal section, with the second plate disposed between the gear nut and the first plate, prior to said positioning the first plate, said positioning the second plate, and said positioning the post.

14. The method of claim 11 wherein driving the external gear teeth of the gear nut comprises removably coupling an instrument to the gear nut and driving the external gear teeth via the instrument to rotate the gear nut.

15. The method of claim 14 wherein removably coupling the instrument to the gear nut comprises moving the instrument toward the gear nut normal the post axis.

16. The method of claim 11 wherein the gear nut comprises a proximal flange that extends farther out away from the post axis than the gear teeth; wherein the proximal flange presses the second plate toward the first plate during said driving the external gear teeth.

17. The method of claim 11 further comprising engaging a fastener with the second plate after the rotating the gear nut and prior to the locking; wherein the locking the second plate comprises advancing the fastener toward the post axis.

18. The method of claim 11 further comprising pivoting the post relative to the first plate prior to said locking.

19. The method of claim 11 further comprising thereafter removing the gear nut from the post by rotating the gear nut in a second direction opposite the first direction.

20. The method of claim 11 wherein displacing the second plate toward the first plate comprises causing protrusions on the first and second plates to bite into opposing lateral sides of adjacent spinous processes.
